(19) United States
(12) Patent Application Publication
(10) Pub. No.: US 2025/0387440 A1
(43) Pub. Date: Dec. 25, 2025

(54) Pharmaceutical Composition for Treatment of Muscle Loss Due to Obesity Treatment

(71) Applicant: Northstrive Biosciences, Inc., Newport Beach, CA (US)

(72) Inventors: Jordan Robert Plews, Sacramento, CA (US); Deniel Mero, Panama City (PA)

(73) Assignee: Northstrive Biosciences, Inc., Newport Beach, CA (US)

(21) Appl. No.: 19/191,209

(22) Filed: Apr. 28, 2025

Related U.S. Application Data
(60) Provisional application No. 63/639,727, filed on Apr. 29, 2024.

(51) Int. Cl. A61K 35/747

(52) U.S. Cl. CPC A61K 35/747

(57) ABSTRACT

The present invention relates to a pharmaceutical composition including a microorganism transformed with a cell surface display vector operably linked with a gene encoding myostatin and activin A proteins for the treatment of muscle loss related to obesity. The pharmaceutical composition may include pharmaceutically viable excipients, such as antiadherents, binders, carriers, coatings, colorants, disintegrants, diluents, flavorings, glidants, lubricants, preservatives, sorbents, or vehicles.

### CROSS-REFERENCE TO RELATED APPLICATIONS

This application claims priority to U.S. Provisional Patent Application Ser. No. 63/639,727, filed Apr. 29, 2024, and titled “Pharmaceutical Composition for Treatment of Muscle Loss Due to Obesity”;
the contents of which are hereby incorporated by reference.

### BACKGROUND

### Field of the Invention

The present invention relates to a treatment for muscle loss due to obesity treatments. More specifically, the present invention relates to a microorganism transformed with a cell surface display vector expressing myostatin and activin A as a bispecific antigen.

### Description of the Related Art

GDF-8 (Growth Differentiation Factor-8), also called myostatin as a growth controlling factor, which selectively negative regulates skeletal muscle growth, is discovered in 1997. A research team, which discovered myostatin, has announced that two high quality cow breeds due to their high muscular mass and tender meat, i.e., Belgian blue and Piedmontese, have mutation in gene encoding myostatin, which results in muscle development, and reported that double-muscle animals of these breeds have average muscle mass increased by 20-25% based on that of ordinary animals.

Experimentally, myostatin-knockout mice also showed significant increases in skeletal muscle mass, and muscles isolated from myostatin-negative mice were about 2- to 3-fold heavier than muscles isolated from wild mice. It has been reported that knockout mice have about 35% higher total body weight than that of wild mice and myostatin-deficient mice have 80% more muscle fibers than that of normal mice, and the increment of skeletal muscle observed in the knockout mice is caused by abnormal growth of muscle fibers as well as an increase in the number of muscle fibers.

Myostatin as a growth controlling factor, which selectively negative regulates skeletal muscle growth, belongs to TGF-α (transforming growth factor-α) super family, is composed of 375 amino acid precursors, and has the same C-terminal fragments of about 109 amino acid residues in mice, rats, human, swine, fowl and turkey and only 3 amino acid residues in the C-terminal region thereof are not the same in monkeys, cows, and sheep. The C-terminal regions are expected to include physiologically active portion of myostatin. Myostatin has shown a high degree of conservation along evolution among various species, which implies that myostatin is an essential factor in biological muscle control.

Myostatin expression is limited to skeletal muscle and it is expressed at low levels in adipose tissue. It seems that myostatin functions as a negative regulator of skeletal muscle growth, but the physiological role of myostatin in an adult individual is not known. Although studies on the physiological role of myostatin have been focused on abnormal growth or its regeneration ability after muscle damage, it is also known that myostatin inhibits adipose tissue growth. However, it has not been known yet whether myostatin acts locally or systemically to regulate animal growth.

Recently, various studies are being conducted on inactivating or inhibiting myostatin exerting the role for negative regulation of skeletal muscle growth. Representative studies thereof include the development of therapeutic agents for treatment of human diseases including muscle-wasting diseases such as muscular dystrophy or muscular atrophy, or muscle loss caused by AIDS, cancer and the like, and an attempt to develop feedstuff additives for growing livestock with high quality meat. Moreover, since, when myostatin was developed as supplement additives for muscle enhancement, it inhibited body fat accumulation due to an increase in the amount of muscle, it is expected to be effective for obesity treatment, and thus, studies thereon are also being conducted.

Two representative studies on a method for inducing muscle growth by inhibiting the function of myostatin protein, are being conducted. One is to discover and use various proteins (follastatin, mutant activin type II receptor, myostatin propeptide, etc.) which inhibit myostatin activity to suppress its function, and the other is to produce antibodies against myostatin polypeptide by animal immunization using myostatin polypeptide, a subsequence thereof, and mutant subsequences.

It was reported that the production of antibodies against myostatin immunogens in vertebrate animals results in a reduction in endogenous myostatin activity, thus showing biological effects such as body weight gain, increased muscle mass, an increase in the number of muscle cells, an increase in muscle cell size, a decrease in the amount of body fat, an increase in muscular strength and the like. However, because the number and type of muscle fibers are genetically programmed during embryogenesis, a decrease in myostatin activity does not lead an increase in the number of muscle fibers in fully grown breeding animals, which may negatively affect meat quality, breed characteristics and fat ratio, but an increase in body weight and growth rate caused by abnormal muscle growth in animals showing a decrease in myostatin activity provides effective methods in the production of beef, pork and poultry meat.

However, most of the studies are conducted by artificially synthesizing myostatin polypeptide or the subsequence thereof or preparing by isolation and purification after expressing them in Escherichia coli, which results in economic inefficiency and thus it is difficult to apply them to industrial applications.

In livestock industry, various breeding programs to enhance the growth rate of animals by increasing feedstuff efficiency are being developed and improved. Among them, medical approaches include methods of administering antibiotics or antibiotic-like compounds to breeding animals or administering hormones such as growth hormones to them. However, administration of antibiotics or antibiotic-like compounds to breeding animals is banned, since it can cause a problem of inducing antibiotic cross-resistance. Additionally, in the case of administration of growth hormone to breeding animals, it is disadvantageous in that it costs a lot, short period of treatment should be repeated because of a short half-time of growth hormone, and growth hormone remaining in meat obtained from animals treated therewith may cause health problems in humans. Because of the difficulties in the medical approaches, various breeding programs to enhance the growth rate of animals by increasing feedstuff efficiency, are being continuously developed.

Technology of expressing by attaching a desired protein to the cellular surface of microorganisms is referred to as a cell surface display technology. The cell surface display technology is to express a foreign protein on the cellular surface using the surface protein of microorganisms, such as bacteria or yeasts, as a surface anchoring motif, and is used in a wide range of applications, including the production of recombinant live vaccines, the construction and screening of peptide/antibody libraries, whole cell absorbents and bioconversion catalysts. The application range of this technology is determined depending on what kind of protein is expressed on the cell surface, thus the industrial application potentiality of the cell surface display technology can be said to be significant.

For successful cell surface display, a surface anchoring motif is most important. Selection and development of a motif capable of effectively expressing a foreign protein on the cell surface is the core of this technology. Accordingly, a surface anchoring motif with the following properties should be selected. First, it should have a secretory signal helping the foreign protein to pass through an intracellular membrane, and to reach to the cell surface. Second, it should have a target signal helping the foreign protein to be stably attached to the outer surface of the cell membrane. Third, it should be expressed on the cell surface at large amounts but has little or no effect on the growth of cells. Fourth, it should be stably expressed regardless of the protein size, without causing a change in the three-dimensional structure of the foreign protein. However, a surface anchoring motif satisfying all the above requirements have not yet been developed.

Cell surface anchoring motifs, which have been known and used till now, are broadly classified into four kinds, i.e., outer membrane proteins, lipoproteins, secretory proteins, and surface proteins such as flagella proteins. In the case of gram-negative bacteria, proteins present in the outer cell membrane, such as LamB, PhoE, and OmpA, were mainly used. Moreover, lipoproteins, such as TraT, PAL (peptidoglycan associated lipoprotein), and Lpp, were also used. Furthermore, the expression of foreign proteins was also attempted using FimA, a fimbriae protein such as the FimH adhesin of type 1 fimbriae or a pili protein such as a PapA pilus subunit as surface anchoring motifs. In addition, it was reported that an ice nucleation protein, pullulanase of Klebsiella oxytoca, IgA protease of Neisseria, E. coli adhesin AIDA-1, VirG protein of Shigella, and a fusion protein of Lpp and OmpA can be used as surface anchoring motifs.

In the case of using Gram-positive bacteria, there is a report that a malaria antigen was effectively expressed using Staphylococcus aureus-derived protein A and FnBPB protein, as surface anchoring motifs. In addition, it was reported that surface coat protein of lactic acid bacteria was used in surface expression and surface protein of Gram-positive bacteria such as a Streptococcus pyogenes-derived M6 protein, S-layer protein EA1 of Bacillus anthracis, Bacillus subtilis CotB, etc., were also used as surface anchoring motifs.

Many studies were performed in an attempt to stably express the antigen or epitope of pathogenic organisms in bacteria where mass production is possible by genetic engineering techniques using the above-described surface anchoring motifs. It was reported that, particularly when a foreign immunogen expressed on the surface of non-pathogenic bacteria is orally administered alive, a more lasting and strong immune response than that of the prior vaccine using attenuated pathogenic bacteria or viruses, can be induced. This induction of the immune reaction is induced by an in vivo immune response to the live bacteria, which is known to be because the surface structures of the bacteria act as adjuvants increasing the antigenicity of the surface-expressed foreign protein. The development of a recombinant live vaccine of non-pathogenic bacteria using this surface expression system is noticeable.

Obesity is a common worldwide public health issue which causes a loss of muscle mass and strength. Obesity can cause biological dysfunction of skeletal muscles leading to atrophy. Current common pharmacological treatments for obesity focus on the reduction of blood glucose to assist obese patients losing weight. None of the currently available pharmacological treatments for obesity target muscle loss and atrophy of skeletal muscle.

Research is underway regarding treatment for sarcopenic obesity, including muscle loss, no currently available treatments target skeletal muscle loss and atrophy within non-aging populations.

### SEQUENCE LISTING XML INCORPORATION BY REFERENCE

A sequence listing XML file BLS-M32.xml is hereby incorporated by reference created on 28 Apr. 2025 at 14:38 with a size of 4,023 bytes (4,096 bytes on disk).

### SUMMARY

The present invention is a pharmaceutical composition including a microorganism transformed with a cell surface display vector expressing myostatin and activin A as a bispecific antigen, where the pharmaceutical composition is used for the treatment of muscle loss due to obesity.

### DETAILED DESCRIPTION OF EMBODIMENTS

In the following description, for purposes of explanation and not limitation, details and descriptions are set forth in order to provide a thorough understanding of the present invention. However, it will be apparent to those skilled in the art that the present invention may be practiced in other embodiments that depart from these details and descriptions without departing from the spirit and scope of the invention.

One embodiment of the present invention is a pharmaceutical composition including a microorganism transformed with a cell surface display vector expressing myostatin and activin A as a bispecific antigen, where the pharmaceutical composition is used for the treatment of muscle loss due to obesity.

The composition may include a pharmaceutically acceptable carrier in addition to the active ingredient, and the carrier is one that is commonly used in the preparation of pharmaceutically acceptable ingredients and includes lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate, and mineral oil, but is not limited thereto. In addition to the above ingredients, the pharmaceutical composition of the present invention may further include lubricants, wetting agents, sweeteners, flavoring agents, emulsifiers, suspending agents, preservatives, and the like. Suitable pharmaceutically acceptable carriers and formulations are well known in the art.

In addition, an appropriate dosage of the pharmaceutical composition according to the present invention may be prescribed diversely depending on factors such as the formulation method, administration method, a patient's age, weight, sex, pathological conditions, diet, administration time, route of administration, excretion rate, and response sensitivity.

In addition, the pharmaceutical composition of the present invention may be administered orally or parenterally, and when administered parenterally, it may be administered by intravenous injection, subcutaneous injection, intramuscular injection, intraperitoneal injection, transdermal administration or the like, and it is preferable that the route of administration is determined depending on the type of disease to which the pharmaceutical composition of the present invention is applied.

In addition, the pharmaceutical composition according to the present invention is formulated using a pharmaceutically acceptable carrier and/or excipient according to a method that may be easily performed by a person skilled in the art to which the present invention pertains, so it may be prepared in the form of a unit dose or prepared by placing it into a multidose container. At this time, the dosage form may be in the form of a solution, suspension, or emulsion in an oil or aqueous medium, or may be in the form of an extract, powder, granule, tablet, or capsule, and may further include a dispersant or stabilizer.

In addition, the pharmaceutical composition according to the present invention may further include carriers and vehicles commonly used in the pharmaceutical field. Specifically, it may include ion exchange resins, alumina, aluminum stearate, lecithin, serum proteins (e.g., human serum albumin), buffering substances (e.g., various phosphates, glycine, sorbic acid, potassium sorbate, partial glyceride mixtures of saturated vegetable fatty acids), water, salts or electrolytes (e.g. protamine sulfate, disodium hydrogen phosphate, calcium hydrogen phosphate, sodium chloride, and zinc salts), colloidal silica, magnesium trisilicate, polyvinylpyrrolidone, cellulosic matrices, polyethylene glycol, sodium carboxymethylcellulose, polyarylate, wax, or lanolin, but is not limited thereto.

In addition, the pharmaceutical composition according to the present invention may be in the dosage forms of granules, powder, coated tablets, tablets, capsules, suppositories, syrups, juices, suspensions, emulsions, drops, injections, or sustained-release dosage forms of an active compound, and it may be administered in various oral or parenteral dosage forms. When formulated, it may be prepared using fillers, extenders, binders, wetting agents, disintegrants, diluents such as surfactants or excipients commonly used in the pharmaceutical field.

In another embodiment of the present invention, the cell surface display vector may include a pgsA gene.

In a further embodiment of the present invention, the myostatin may have a gene sequence of SEQ ID NO: 1.

In yet another embodiment of the present invention, the activin A may have a gene sequence of SEQ ID NO: 2.

In further embodiments, the microorganism may be a lactic acid bacterium, such as Lactobacillus paracasei.

What is claimed is:

1. A pharmaceutical composition, comprising a microorganism transformed with a cell surface display vector operably linked to a gene encoding myostatin and activin A proteins, wherein the microorganism is used for the treatment of muscle loss related to obesity treatments.

2. The pharmaceutical composition of claim 1, wherein the cell surface display vector includes a pgsA gene.

3. The pharmaceutical composition of claim 1, wherein the myostatin has a gene sequence of SEQ ID NO: 1.

4. The pharmaceutical composition of claim 1, wherein the activin A has a gene sequence of SEQ ID NO: 2.

5. The pharmaceutical composition of claim 1, wherein the microorganism is a lactic acid bacterium.

6. The pharmaceutical composition of claim 5, where in the lactic acid bacterium is Lactobacillus paracasei.

7. The pharmaceutical composition of claim 1, further comprising at least one pharmaceutically viable excipient.

8. The pharmaceutical composition of claim 7, wherein the at least one pharmaceutically viable excipient is selected from the group consisting of antiadherents, binders, carriers, coatings, colorants, disintegrants, diluents, flavorings, glidants, lubricants, preservatives, sorbents, and vehicles.


SEQUENCE LISTING (2 sequences)

SEQ ID NO: 1 - DNA, 387 bp, unidentified
  source 1..387: mol_type = unassigned DNA
ggtggttcaagatctgtcgacgaagtcaaggtaacagacacaccgaaaagatccaggagagattttggactcgactgtgatgagcactcaacagaatctcgatgctgtcgttaccctctaactgtggattttgaagcttttggatgggactggattattgcacccaaaagatataaggccaattactgctctggagagtgtgaatttgtatttttacaaaaataccctcacactcatcttgtgcaccaagcaaaccccagaggttcagcaggcccctgctgtactcccacaaagatgtctccaatcaatatgctatattttaatggcaaagaacaaataatatatgggaaaattccagccatggtagtagatcgctgtgggtgctca

SEQ ID NO: 2 - DNA, 714 bp, unidentified
  source 1..714: mol_type = unassigned DNA
ggtttggaatgtgatggtaaagttaatatttgttgtaaaaaacaattttttgttagttttaaagatattggttggaatgattggattattgctccaagtggttatcatgctaattattgtgaaggtgaatgtccaagtcatattgctggtaccagtggtagtagtttgagttttcatagtaccgttattaatcattatcgtatgcgtggtcatagtccatttgctaatttgaaaagttgttgtgttccaaccaaattgcgtccaatgagtatgttgtattatgatgatggtcaaaatattattaaaaaagatattcaaaatatgattgttgaagaatgtggttgtagtggtggttcaagatccggtttggaatgtgatggtaaagttaatatttgttgtaaaaaacaattttttgttagttttaaagatattggttggaatgattggattattgctccaagtggttatcatgctaattattgtgaaggtgaatgtccaagtcatattgctggtaccagtggtagtagtttgagttttcatagtaccgttattaatcattatcgtatgcgtggtcatagtccatttgctaatttgaaaagttgttgtgttccaaccaaattgcgtccaatgagtatgttgtattatgatgatggtcaaaatattattaaaaaagatattcaaaatatgattgttgaagaatgtggttgtagttaa